(19) United States
(12) Patent Application Publication
(10) Pub. No.: US 2015/0276715 A2
(43) Pub. Date: Oct. 1, 2015

(54) Short-Term Infarction-Based Test for Investigative Drugs

(71) Applicant: Harold Richard Hellstrom, Fayetteville, NY (US)

(72) Inventor: Harold Richard Hellstrom, Fayetteville, NY (US)

(21) Appl. No.: 14/182,272

(22) Filed: Feb. 17, 2014

Related U.S. Application Data
(63) Continuation-in-part of application No. PCT/US2013/048502, filed on Jun. 28, 2013.
(60) Provisional application No. 61/666,857, filed on Jun. 30, 2012.
(60) Provisional application No. 61/691,779, filed on Aug. 21, 2012.

(51) Int. Cl. G01N 33/50; A61K 45/06; A61K 39/395; A61K 31/421

(52) U.S. Cl. CPC G01N 33/5038; A61K 31/421; A61K 45/06; A61K 39/3955

(57) ABSTRACT

Provided herein is a short-term myocardial infarction based test for identifying a compound, substance or drug that reduces the risk of myocardial infarction and optionally ischemic heart disease in a test subject(s). Further provided is a method for preventing or treating myocardial infarction using the compound, substance or drug identified.

### TECHNICAL FIELD

Provided herein is a short-term myocardial infarction based test for identifying a compound, substance or drug that reduces the risk of myocardial infarction in a test subject(s). Further provided is a method for preventing or treating myocardial infarction using the compound, substance or drug identified.

### BACKGROUND

Many new drugs are currently in development which are intended to prevent myocardial infarctions. In addition, it is important to determine whether other investigational drugs could potentially cause myocardial infarctions in recipients as an unintended side effect.

Current clinical trial protocols for investigational drugs are lengthy, involve a large number of participants, and are extremely expensive. Specifically, these tests take many years, involve thousands of participants, and may cost millions of dollars to complete.

Accordingly, there is a need for a short-term clinical trial protocol which enables identification, in a shorter time frame and at a lesser cost, of candidate drugs which are designed to prevent myocardial infarctions. There is also a need for a clinical trial designed to determine the risk of drugs for causing myocardial infarctions in subjects, e.g., over a short time period.

### SUMMARY

The present invention relates to a method of designing a short-term myocardial infarction-based test (e.g., clinical trial) in order to demonstrate if a test compound reduces or increases the risk of myocardial infarctions in a test subject. The methods of the present invention can also detect, in a short time frame, whether a candidate drug has a significant risk of causing myocardial infarction in a test subject.

Trials designed according to the methods of the present invention are advantageous in comparison to traditional Phase III clinical trials for several reasons. First, the trials of the invention are designed to involve fewer participants than standard clinical trials (for example, less than 500 participants). The trials of the invention are also comparatively inexpensive (due to the short time-frame needed to complete the study and the need for fewer participants). They also protect controls from myocardial infarctions, and, importantly, they provide short-term myocardial infarction-based results regarding whether the investigational drug can reduce the risk of myocardial infarction. It is anticipated that additional, long-term clinical trials will still be needed to test for other drug-induced problems, such as side effects and safety considerations.

In one aspect of the invention, a trial designed according to the invention comprises a modification and improvement of known studies of percutaneous coronary interventions (PCI) and acute coronary syndromes (ACS/infarctions). Prior studies show that the acute treatment of PCI and ACS/infarctions by statins can significantly reduce the incidence of periprocedural myocardial infarctions (PCI) and significantly reduce short-term mortality (ACS/infarctions).

The method may comprise:

(a) administering the investigative drug and optionally a statin to a test group of subjects, wherein the test group comprises subjects who are undergoing elective PCI or have ACS/acute myocardial infarction;

(b) administering a statin to a control group of subjects, wherein the test group has been administered an investigative drug and statin or administering a placebo to a control group of subject wherein the test group has been administered an investigative drug alone, wherein the control group comprises subjects who are undergoing elective PCI or have ACS/acute infarction;

(c) comparing the subsequent incidences of myocardial infarctions in the subjects who had PCI in the test group with those had PCI in the control group, and

(d) comparing the subsequent incidences of short-term mortality and/or status of myocardial infarctions in the subjects who had ACS/acute infarction in the test group with that of those who had ACS/acute infarction in the control group;

wherein if there are significantly less incidences of myocardial infarctions in the subjects who had PCI as compared to those in the control group and/or significantly less incidences of short-term mortality and/or improved status of myocardial infarction in the subjects who had ACS/acute myocardial infarction in the test group as compared to those in the control group, then the investigative drug is capable of preventing myocardial infarctions and

wherein if there are significantly more incidences of myocardial infarctions in the subjects who had PCI as compared to those in the control group and/or significantly more incidences of short-term mortality in the subjects who had ACS/acute myocardial infarction and/or if status of myocardial infarction has deteriorated in the test group as compared to those in the control group, then the investigative drug increases the risk of a myocardial infarction.

Thus, in one aspect of the invention, a study or test group comprised of individuals who will be undergoing PCI in the near term (e.g., elective PCI), or are suffering from ACS/infarction (referred to as participants) are administered an investigative drug and optionally a statin. A control group is administered a statin alone, which represents the currently accepted course of therapy or alternatively a placebo when the test group has only been administered the investigative drug alone. If the combination of the statin and the investigative drug is significantly more effective than the statin alone (as administered in the control group) in preventing myocardial infarction and/or acute mortality and/or improving the status of myocardial infarction, the investigative drug is determined to be capable of reducing the risk of myocardial infarction. In another embodiment, if an investigative drug is significantly more effective than the placebo (as administered in the control group) in preventing myocardial infarction and/or acute mortality and/or improving the status of myocardial infarction in a test subject, the investigative drug is determined to be capable of reducing the risk of myocardial infarction. As will be described infra, status of myocardial infarction may be determined by measuring the level of cardiac enzymes in test and control subjects. If significantly more myocardial infarctions occur in the test group, and/or if status of myocardial infarction in a test subject has deteriorated, it is likely that the investigative drug increases the risk of myocardial infarction. The results of clinical trials designed according to the invention are available in the short-term since the participants (those undergoing PCI or having ACS/infarction) are at acute risk for a myocardial infarction or acute mortality. For example, and not by way of limitation, individual results are available in about one, two, or three days for subjects who have undergone PCI and for an individual study of ACS/infarction, results are available in about one, two, or three weeks. In one embodiment, the invention provides relatively prompt overall results, e.g., in six months or less, with multiple study centers.

The present invention is also directed to methods of reducing the risk of myocardial infarction using a drug identified by the methods disclosed herein and optionally in combination with another substance used to reduce the risk of myocardial infarction.

The present invention is also directed to a method for modulating and/or treating myocardial infarction using a drug identified by the methods disclosed herein and optionally in combination with another substance used to modulate and/or treat myocardial infarction by administering an amount of the identified drug and optionally other substance effective to modulate and/or treat myocardial infarction. In a particular embodiment, the method may comprise:
(a) identifying a drug capable of modulating and/or treating infarctions and ischemic heart disease comprising:
   (i) administering an investigative drug and optionally a statin to a test group of subjects, wherein the test group comprises subjects who are undergoing elective PCI or have ACS/acute myocardial infarction;
   (ii) administering a statin to a control group of subjects, wherein the test group has been administered an investigative drug and statin or administering a placebo to a control group of subject wherein the test group has been administered an investigative drug alone, wherein the control group comprises subjects who are undergoing elective PCI or have ACS/acute infarction;
   (iii) comparing the subsequent incidences of myocardial infarctions in the subjects who had PCI in the test group with those had PCI in the control group, and
   (iv) comparing the subsequent incidences of short-term mortality and/or status of myocardial infarction in the subjects who had ACS/acute infarction with that of those who had ACS/acute infarction in the control group;
wherein if there are significantly less incidences of myocardial infarctions in the subjects who had PCI in the test group as compared to those in the control group and/or significantly less incidences of short-term mortality and/or improved status of myocardial infarctions in the subjects who had ACS/acute myocardial infarction in the test group as compared to those in the control group, then the investigative drug is capable of preventing myocardial infarctions.

(b) administering said identified drug in an amount effective to modulating and/or treating infarctions and ischemic heart disease.

### Definitions

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

As defined herein, the term “modulate” means adjusting the frequency and/or severity of myocardial infarction.

As defined herein, the terms “treat”, “treatment” and “treating” are to be understood accordingly as embracing prophylaxis and treatment or amelioration of symptoms of disease as well as treatment of the cause of the disease.

“Percutaneous coronary interventions (PCI),” commonly known as coronary angioplasty or simply angioplasty, is typically used in two clinical situations. Firstly, elective PCI is used to reduce coronary stenoses (narrowed coronary arteries of the heart) by using balloon dilation. Secondly, it is used for the acute treatment of those with ACS/infarction. Here the goal is to remove thromboses, and also to reduce coronary stenoses to allow more blood flow.

“Acute coronary syndrome (ACS)” refers to a spectrum of clinical presentations ranging from those for ST-segment elevation myocardial infarction (STEMI) to presentations found in non-ST-segment elevation myocardial infarction (NSTEMI) or in unstable angina. In terms of pathology, ACS is almost always associated with rupture of an atherosclerotic plaque and partial or complete thrombosis of the infarct-related artery. In some instances, however, stable coronary artery disease (CAD) may result in ACS in the absence of plaque rupture and thrombosis, for example, when physiologic stress (e.g., trauma, blood loss, anemia, infection, tachyarrhythmia) increases demands on the heart. The diagnosis of acute myocardial infarction in this setting requires a finding of the typical rise and fall of biochemical markers of myocardial necrosis in addition to at least one of the following: ischemic symptoms: development of pathologic Q waves, or ischemic ST-segment changes on electrocardiogram (ECG) or in the setting of a coronary intervention.

A “statin” is a member of a broad class of compounds that inhibit the activity of the enzyme 3-hydroxy-3-methylglutaryl coenzyme A (HMG-CoA) reductase. Examples of statins that may be used in connection with the subject methods include, but are not limited to, lovastatin; simvastatin; pravastatin sodium; fluvastatin sodium; atorvastatin; rosuvastatin; and pitatvastatin.

### DETAILED DESCRIPTION

The present invention is based on the discovery of a short-term myocardial infarction-based test (clinical trial) which is designed to demonstrate if a test compound, e.g., a CETP inhibitor, PCSK9 agent or other investigational drug, reduces the risk of myocardial infarction in a test subject.

CETP inhibitors are members of a class of drugs that inhibit cholesteryl ester transfer protein (CETP). They are intended to reduce the risk of atherosclerosis by improving blood lipid levels. Cholesteryl ester transfer protein normally transfers cholesterol from high density lipoprotein (HDL) cholesterol to very low density or low density lipoproteins (VLDL or LDL). Inhibition of this process results in higher HDL levels (the so-called “good” cholesterol-containing particle) and reduces LDL levels (the so-called “bad” cholesterol). Examples of CETP inhibitors currently under development include anacetrapib (Merck) and evacetrapib (Eli Lilly & Company). The development of torcetrapib (Pfizer), another CETP inhibitor, was halted in 2006 when phase III studies showed excessive all-cause mortality in the treatment group receiving a combination of atorvastatin (Lipitor) and torcetrapib.

A “PCSK9 agent” is an agent that modulates the expression and/or synthesis of PCSK9 (proprotein convertase subtilisin kexin 9). PCSK9 is a member of the subtilisin serine protease family that is involved with regulation of hepatic LDL receptor activity. PCSK9 agents may include but are not limited to the PCSK9 monoclonal antibodies, peptide mimics and anti-sense oligonucleotides. PCSK9 agents have been developed to prevent myocardial infarctions by lowering low-density lipoprotein (LDL) cholesterol. These agents generally are planned to be used to supplement statins. PCSK9, a secreted protease, is involved with regulation of hepatic LDL receptor activity ^{41,42}Blocking PCSK9 binding to the LDL receptor with a monoclonal antibody lowers LDL cholesterol in humans.^{41,42}

Trials designed according to the present invention also detect if investigational or candidate drugs increase the risk of myocardial infarction in a test subject. The present invention is also directed to methods of reducing the risk of myocardial infarctions using a drug identified by the trials designed according to the methods disclosed herein. The participants used in the trials designed according to the methods of the present invention include subjects who will be undergoing elective percutaneous coronary interventions (PCI) or are suffering from acute coronary syndromes (ACS/infarctions). The standard treatment of elective PCI and ACS/infarction by administering a statin is incorporated into the invention. The test group is given a statin plus the experimental drug, and the control group is given only a statin. Alternatively, the test group may be given the experimental drug and the control group is given only a placebo. In a particular embodiment, the test group and control group may be about the same size.

As described in more detail below, it has been estimated that approximately 40-50% of individuals who have undergone PCI have a mild myocardial infarction.^{1,2} The accepted course of treatment during and after PCI involves administration of statins, which prevents about half of these myocardial infarctions. Therefore, it would be expected that approximately 25% of these patients will still experience a myocardial infarction, even when given a statin, in the days following the initial episode. With ACS/myocardial infarction, there is a significant short-term mortality.

The invention described herein describes a clinical trial wherein patients who will undergo elective PCI, or have ACS/infarction and are in need of therapy, would be given the experimental drug in addition to the normally administered statin (the test group). A control group is administered statin alone.

The results (e.g., the ability of the test compound to inhibit myocardial infarctions in the PCI group and inhibit mortality in the ACS/infarction group) are available in the short-term since the participants (those having undergone PCI or had ACS/infarction) are at acute risk for a myocardial infarction or acute mortality. For example, individual results are available in about one, two, or three days for subjects who have undergone PCI. For an individual study of ACS/infarction, results are available in about one, two or three weeks. In one embodiment, the invention provides relatively prompt overall results, e.g., in six months or less, with multiple study centers. Likewise, an increase of myocardial infarctions caused by the administration of the experimental drug is also apparent in the short-term.

### Basis for the Short-Term Myocardial Infarction-Based Test of the Invention

The invention is based on the principles that statins and the investigative drug operate additively and acutely by one basic mechanism. Therefore, without being bound by any particular theory, it is useful to list these specific principles, as follows:

### 1. Risk Factors Directly Induce Myocardial Infarction by Expression of Thrombosis/Vasoconstriction

It seems apparent that risk factors directly induce myocardial infarction by expression of thrombosis/vasoconstriction; after all, thrombosis is the accepted,³ and spasm is a proposed,⁴ mechanism for the direct induction of myocardial infarction. There is clear evidence that multiple and diverse risk factors for ischemic hear disease (IHD) (pharmaceutical and lifestyle) express as thrombosis/vasoconstriction.⁵ Risk factors favor thrombosis/vasoconstriction through endothelial dysfunction and a separate tendency toward thrombosis such as platelet activation and/or sympathetic activation.⁵ Myocardial infarctions associated with COX-2 inhibitors provide a specific example of the direct induction of myocardial infarction by risk factor-induced thrombosis/vasoconstriction and are generally attributed directly to thromboxane—which expresses thrombosis/vasoconstriction.⁶ This evidence is of particular importance to the short-term test of the invention, as it is based on a drug.

### 2. Thrombosis or Vasoconstriction and Anti-Thrombosis or Vasodilation

For the purpose of the short-term myocardial infarction-based test, no opinion is taken about whether thromboses or vasoconstriction directly induce myocardial infarctions—or whether anti-thrombosis or vasodilation directly prevents myocardial infarctions. The distinction is not relevant to this invention. Thrombosis/vasoconstriction (and anti-thrombosis/vasodilation) tends to occur together as a unit⁵—and thromboses is the accepted,³ and spasm a proposed,⁴ mechanism for myocardial infarction.

### 3. Preventative Factors, Pharmaceutical and Lifestyle, Prevent Myocardial Infarctions Through Expression of Anti-Thrombosis/Vasodilation

There is clear evidence that multiple and diverse pharmaceutical and lifestyle preventative factors for IHD express anti-thromboses/vasodilation⁵—as part of pleiotrophic effects. If thromboses/vasoconstriction causes myocardial infarctions, reasonably, anti-thrombosis/vasodilation prevents myocardial infarctions.

Importantly, it generally is accepted that aspirin prevents myocardial infarctions through anti-thrombosis⁷—which reflects the standard paradigm that myocardial infarctions are due directly to thromboses. Aspirin inhibits platelets, which express thrombosis/vasoconstriction.⁷

There is inferential evidence that statins prevent myocardial infarctions through anti-thrombosis/vasodilatory effects. Endothelial dysfunction favors thrombosis/vasoconstriction,^{5,8,9} and statins improve endothelial dysfunction.¹⁰⁻¹⁴ Statins also depress the thrombotic arm of thrombosis/vasoconstriction.^{15,16} Further, statins suppress COX-2 inhibitors¹⁷—which express thrombosis/vasoconstriction.^{7,18}

Other pharmaceutical preventative agents for IHD improve thrombosis/vasoconstriction. Significantly, aspirin¹⁹ and angiotensin-converting enzyme inhibition²⁰ also improve endothelial dysfunction. In general, multiple pharmaceutical and lifestyle preventative factors express pleiotrophic effects, which expresses anti-thrombosis/vasodilation.⁵

### 4. Risk Factors Act Acutely to Induce Myocardial Infarction (Through Thrombosis/Vasoconstriction)

There is evidence that risk factors act acutely to induce myocardial infarction. Significantly, 82.2% of myocardial infarctions in one series acutely followed “triggering” risk factors as acute stress, a heavy meal, and “Monday.”²¹ This is interpreted as evidence that multiple risk factors (which express thrombosis/vasoconstriction) can act acutely. Also significant, mental stress induced transient endothelial dysfunction (which favors thrombosis/vasoconstriction) in thirty minutes.²²

### 5. Preventative Agents can Prevent Myocardial Infarctions Promptly (Supposedly Through Anti-Thrombosis/Vasodilation)

There is convincing evidence that acute statin therapy promptly reduces the incidence of periprocedural myocardial infarctions after percutaneous coronary interventions (PCI), and reduces the incidence of short-term mortality with acute coronary syndromes (ACS/infarction). The multiple studies of PCI and ACS treated with acute statin therapy show very impressive results—usually around or better than a 50% improvement.

Individual studies of PCI showed a significant reduction of periprocedural myonecrosis with statins over controls by 3.7% vs. 9.4%,²³ 9.5% vs. 15.8%,²⁴ and 5% vs. 18%.¹ Also, the incidence of large non-Q-wave myocardial infarction was 8% in the statin group and 15.6% in the control group.²⁵

Meta-analyses of statin treatment with PCI showed similar results. There was a reduction of periprocedural myonecrosis over controls of 9.0% vs. 17.5%² and 7.7% vs. 14.2%.²⁶ Another large study showed a 43% reduction of post-procedural myocardial infarctions.²⁷

Studies of acute statin use with ACS/infarction showed reduction of in-hospital mortality and morbidity as compared to controls by 4.0-5.3% vs. 15.45²⁸ and 5% vs. 17%.²⁹

Meta-analyses of statin use with ACS/infarctions showed a reduction of deaths at 7 days (0.4% vs. 2.6%)³⁰ and 30 days (0.5% vs. 1.0%).³¹

Finally, acute statin therapy with PCI in cases of ACS showed a lower rate of periprocedural myocardial injury over controls (5.8% vs. 11.4%).³²

There is evidence that statins act acutely to prevent myocardial infarctions through anti-thrombosis/vasodilatory effects; statins improved endothelial dysfunction (which favors thrombosis/vasoconstriction) when measured at 60 minutes,¹⁰ 24 hours, ¹¹ 10 days,¹² 2 weeks,¹³ and 4 weeks.¹⁴ Also, anti-platelet effects (anti-thrombosis/vasodilation) of aspirin are measurable by 60 minutes.⁷

Further, angiotensin-converting inhibition improved endothelial dysfunction when measured at 4 weeks.²⁰ Another study³³ showed that angiotensin converting enzyme inhibition prompted parasympathetic activation (which improves endothelial dysfunction⁵) when measured at 30 days. While measured at a month's time, it seems reasonable that actual benefits occurred significantly earlier.

The lability_of endothelial function can be used as evidence that preventative substances tend to act promptly to improve endothelial dysfunction. This liability is demonstrated by several parameters: The ability of mental stress to induce transient endothelial dysfunction by 30 minutes,²² the ability of statins to promptly improve endothelial dysfunction, and the very beneficial effects of acute statin therapy with PCI and ACS/infarction. In this light, it is likely that angiotensin-converting enzyme inhibition improved endothelial dysfunction much more promptly than 4 weeks. It also is likely that other pharmaceutical agents that prevent myocardial infarction and improve endothelial function act acutely.

### 6. Risk Factors Act Additively

There is general agreement that risk factors act additively.^{3,34}

### 7. Preventative Pharmaceutical Agents Operate Additively

That preventative agents act additively is commonly accepted.³ As example, the combination of statins, angiotensin converting inhibitors, and aspirin reduced the risk of death in IHD by 71%.³⁵

### 8. Summary

Again, without wishing to be bound by any particular theory, the above evidence supports the tenet that preventative pharmaceutical agents operate acutely and additively, most likely by pleiotrophic anti-thrombosis/vasodilation. Therefore, the short-term myocardial infarction-based test for investigative drugs is based on sound principles. If the combination of a preventative measure (especially a statin) plus an investigative drug act significantly more beneficially than a preventative measure (e.g., a statin), this is evidence that the investigative drug reduces the risk of myocardial infarction.

A second rationale can be used: as the preventative agent (as a statin) is given to both the test and control groups, the preventative agent cancels out. Therefore, the test evaluates the ability of the investigative drug to act more beneficially than the control group.

### Methods of Designing Clinical Trials of the Invention

In one aspect of the present invention, individuals who are undergoing elective PCI or are experiencing ACS/acute myocardial infarction are separated into two groups, the test group and the control group. In a particular embodiment, the test group and the control group may be about the same size. The test group is given a statin plus the investigative drug, and the control group is given the usual statin (e.g., at about the same dose as the test group). In a particular embodiment, statin therapy is given according to standard protocols for the treatment of elective PCI and ACS/acute myocardial infarction.

Generally, the investigative drug is given at the same time as the statin. Alternatively, as set forth above, the test group is given the investigative drug and the control group is given a placebo.

If there is a statistically significant lower incidence of myocardial infarction (for the subjects who are undergoing elective PCI) and short-term mortality (for the subjects with ACS/acute myocardial infarction) in the test group, this is prima facie evidence that the investigative drug reduces the risk of myocardial infarctions when used in the usual clinical setting. Preferentially, statistically significant results should include about a 10% or more reduction of infarctions between about one day to about seven days after undergoing the elective PCI and/or about a 10% or more reduction in mortality rate between about two weeks to about one month, two months, three months, four months, five months or six months after undergoing the elective PCI. However, if the test group has a pronounced higher incidence of myocardial infarctions or mortality, it is likely that the drug causes myocardial infarctions. By giving all test group participants a statin, including the control group, all cases are treated as any individuals undergoing elective PCI or treatment of ACS/acute myocardial infarction would be treated under the current standard of care. Because of the administration of the statin, both the test group and the control group are protected against myocardial infarction. As both the test and control groups are given about the same dose of a statin, in some embodiments, effects of the statin are balanced out, leaving only the effect of the experimental drug on PCI and ACS/infarction.

The design of the clinical trials of the invention provide relatively prompt results as compared to accepted clinical trials of investigational drugs. In one embodiment, results of individual cases should be available, for example, in about one day, two, three, four, five, or six days, or a week for those who had elective PCI (measuring post-procedural myocardial infarctions), and within about a week, two weeks, three weeks, or a month, for those who had ACS/infarctions (measuring short-term mortality).

Also, because myocardial infarctions are highly concentrated, relatively small numbers of test subjects are necessary, e.g., about 50, 100, 200, 300, 400 or 500 test subjects, for the methods of the invention. For individuals who have suffered from ACS/infarction, about 100% of cases have myocardial infarctions. With PCI, incidence of periprocedural myocardial infarctions up to 40-50% have been reported.^{1,2} However, studies of PCI reported above showed incidences of periprocedural myocardial infarctions in controls between 9.4%,²³ 15.6%,²⁵ 15.7%,²⁴ and 18%.¹ Therefore, smaller numbers of cases are needed with ACS/infarction than with PCI to achieve statistical significance. However, total number of combined controls and test cases for PCI have been rather small (153,¹668,²⁴ 383,²³ and 451²⁵).

Periprocedural myocardial infarctions generally are mild and only detected by elevation of cardiac enzymes.^{1,24} However, these mild infarctions are treated conventionally as genuine mild infarctions. In keeping with this, the incidence of large non-Q-wave infarction after PCI was 8% in the statin group and 15.6% in the control group²⁵—findings similar to studies of periprocedural myonecrosis after PCI.

However, to solidify that the short-term myocardial infarction-based test directly predicts results of standard long-term phase III tests, it is helpful to use both PCI and ACS models. The former model is based on preventing myocardial infarctions, and the latter model is based on reducing the impact of an acute ACS/infarction.

In some embodiments, dosage of statins for the short-term myocardial infarction-based test of the invention follows common practices with statin treatment of elective PCI and with ACS/infarction. The patient, in a specific embodiment, may be administered low (10-20 mg), moderate (20-40 mg) or high doses (40-80 mg) of statin. For example, 80^{23,24} and 40¹ mg per day of atorvastatin has been used with elective PCI and can be used in the methods of the invention, although use of moderate doses of other statins is not excluded. For example, in an alternative embodiment, a “high” dose of 40 mg, “moderate” dose of 20 mg, “low” dose of 10 mg and “very low” dose of 5 mg of rosuvastatin may be used. Also, in one embodiment, the statin therapy can be done in combination with one or more other effective preventative agents, such as angiotensin-converting enzyme inhibitors. Also, use of preventative drugs other than statins are also included in some embodiments.

Although there is evidence that the acute effects of statins are manifested quickly in favoring anti-thrombosis/vasodilation, in one embodiment, there can be a period of pretreatment, for example to ensure full activation of the investigative drug. Pretreatment with statins (and the investigative drug) for elective PCI can be, for example, 12 hours to 31 days or more in advance. For example, pretreatment times of statins for PCI have ranged from around 12 hours²³ to 31 days,² and most times have been about 7 days or more.^{1,2,24} Common practices for advance administration of the statin can be used in the methods of the invention.

The investigative drug can also be administered in advance of PCI. If there is concern that the investigative drug will take longer than statins to develop its full therapeutic effect, the dosing of the investigative drug for elective PCI can begin significantly longer than a week prior to PCI. For ACS, to account for a possible tardy full effect of the investigative drug, evaluation of short-term mortality can be extended past 4 weeks, for example to 6 weeks or 8 weeks.

Doses of an investigative drug can be employed as used in other trials of the investigative drug or as determined by pre-clinical trials or determined based on dose of other like drugs. In general, investigative drugs can be used at high dosage, but moderate doses are not excluded.

Differences of incidences of periprocedural infarctions (PCI) and short-term mortality (ACS/infarction) between the test and control group are determined, using appropriate statistical methodology as is known in the art.

Incidences of periprocedural myocardial infarctions with PCI is determined in test and control groups by standard methods for determining the occurrence of myocardial infarction.³⁶ In one embodiment, biomarker evaluation of myocardial infarction can be used.^{36,37} The preferred biomarker for myocardial necrosis is cardiac troponin (I or T).^{36,37} With PCI, in one embodiment, measurement of cardiac enzymes is performed before or immediately after the procedure, and again at 6-12 and 18-24 hours.³⁶

For ACS/infarction, in one embodiment, evaluation of the status of the myocardial infarction in test and control cases (especially by cardiac enzymes) is performed at admission, several times during the hospital stay, and when the protocol ends the trial, for example, at one week, one month, or six weeks. In particular, status of myocardial infarction may be determined to be improved in test subjects if there is a statistically significant reduction in cardiac enzymes in test subjects as compared to controls.

### CETP Inhibitors

Current investigative cholesteryl ester transfer protein (CETP) inhibitor drugs (for example, anacetrapib (Merck) and evacetrapib (Eli Lilly & Company)) and other similar drugs are especially propitious drugs for testing by the short-term myocardial infarction-based test of the invention. The proposed uses of these drugs simulates the short-term myocardial infarction-based test.

These drugs, which elevate high density lipoprotein (HDL) cholesterol,³⁸⁻³⁹ are generally planned to be used to supplement drugs (as statins) which lower low density lipoprotein (LDL) cholesterol.

Trials of experimental CETP inhibitors used the following doses: anacetraapib 100 mg/day³⁸ and evacetrapib 30, 100, and 500 mg/day.⁴⁰

### PCSK9 Agents

PCSK9 agents have been developed to prevent myocardial infarctions by lowering low-density lipoprotein (LDL) cholesterol. These agents may be used to supplement statins but could be used alone as well. PCSK9, a secreted protease, is involved with regulation of hepatic

LDL receptor activity^{41,42}

Blocking PCSK9 binding to the LDL receptor with a monoclonal antibody has been found to lower LDL cholesterol in humans.^{41,42} The PCSK9 monoclonal antibody AMG 145 (Amgen) has been injected subcutaneously every four weeks at 350 mg. and 420 mg.^{41,42} as well as subcutaneously every 2 weeks at 70 mg., 105 mg., and 140 mg. Also, the monoclonal antibody REGN727/SAR236553 (Regeneron/Sanofi) was injected subcutaneously every 4 weeks at doses of 200 or 300 mg., or 150 mg every two weeks.^{41,42}

### Advantages of the PCI and ACS/Myocardial Infarction Models

There are advantages to both the PCI and the ACS/acute infarction models. Elective PCI allows premedication. Also, periprocedural infarctions after PCI generally are mild,^{1,24} thus limiting the risk of the study. Post PCI myocardial infarctions generally are asymptomatic,¹ and generally are defined as a three fold elevation of creatine kinase-myocardial isoenzyme.²⁴ Also, if, as expected, the investigative drug reduces the risk of myocardial infarctions, this will aid half the cases (the test group).

The ACS/acute myocardial infarction model has a special advantage, as myocardial infarctions are serious and can result in significant short-term mortality. If the investigative drug reduces the risk of myocardial infarctions, the drug will give more protection against short-term mortality to half the cases (the test group).

An important issue is the ability of the investigative drug to fare well with the test. That is, to prevent myocardial infarctions with individuals undergoing PCI, and lower short-term mortality with individuals suffering from ACS. Importantly, the short-term myocardial infarction-based test of the invention simulates two separate clinical situations: the direct prevention of myocardial infarctions (with individuals undergoing PCI) and limiting the acute term mortality of myocardial infarctions (with individuals suffering from ACS/infarctions).

If an investigative drug is effective in these situations, the drug will likely prevent infarctions in the clinical situation in high risk individuals in a clinical setting.

### REFERENCE LIST

1. Pasceri V, Patti G, Nusca A, Pristipino C, Richichi G, Di Sciascio G et al. Randomized trial of atorvastatin for reduction of myocardial damage during coronary intervention: results from the ARMYDA (Atorvastatin for Reduction of Myocardial Damage during Angioplasty) study. Circulation 2004;110:674-78.
2. Merla R, Reddy N K, Wang F W, Uretsky B F, Barbagelata A, Birnbaum Y. Meta-analysis of published reports on the effect of statin treatment before percutaneous coronary intervention on periprocedural myonecrosis. Am J Cardiol 2007;100:770-76.
3. National Cholesterol Education Program. Third report of the expert panel on detection, evaluation and treatment of high blood cholesterol in adults (Adult Treatment Panel III). Circulation 2002;106:3143-421.
4. Hellstrom H R. Can the premises of the spasm of resistance vessel concept permit improvement in the treatment and prevention of ischemic heart disease? Med Hypotheses 2003;60:36-51.
5. Hellstrom H R. The altered homeostatic theory: a hypothesis proposed to be useful in understanding and preventing ischemic heart disease, hypertension, and diabetes—including reducing the risk of age and atherosclerosis. Med Hypotheses 2007;68:415-33.
6. FitzGerald G A. Coxibs and cardiovascular disease. N Eng J Med 2004;351:1709-11.
7. Awtry E H, Loscalzo J. Aspirin. Circulation 2000;101:1206-18.
8. Fuster V, Moreno P R, Fayad Z A, Corti R, Badimon J J. Atherothrombosis and high-risk plaque: part I: evolving concepts. J Am Coll Cardiol 2005;46:937-54.
9. Brunner H, Cockcroft J R, Deanfield J, Donald A, Ferrannini E, Halcox J et al. Endothelial function and dysfunction. Part II: Association with cardiovascular risk factors and diseases. A statement by the Working Group on Endothelins and Endothelial Factors of the European Society of Hypertension. J Hypertens 2005;23:233-46.
10. Ghaffari N, Ball C, Kennedy J A, Stafford I, Beltrame J F. Acute modulation of vasoconstrictor responses by pravastatin in small vessels. Circ J 2011;75:1506-14.
11. Wassmann S, Faul A, Hennen B, Scheller B, Böhm M, Nickenig G. Rapid effect of 3-hydroxy-3-methylglutaryl coenzyme a reductase inhibition on coronary endothelial function. Circ Res, 2003;93:e98-e103.
12. Karatzis E, Lekakis J, Papamichael C, Andreadou I, Cimponeriu A, Aznaouridis K et al. Rapid effect of pravastatin on endothelial function and lipid peroxidation in unstable angina. Int J Cardiol 2005;101:65-70.
13. John S, Dellas C, Jacobi J, Schlaich M P, Schneider M, Schmitz G et al. Rapid improvement of nitric oxide bioavailability after lipid-lowering therapy with cerivastatin within two weeks. J Am Coll Cardiol 2001;37:1351-58.
14. O'Driscoll G, Green D, Taylor R R. Simvastatin, an HMG-coenzyme A reductase inhibitor, improves endothelial function within 1 month. Circulation 1997;95:1126-31.
15. Undas A, Brummel K E, Musial J, Mann K G, Szczeklik A. Simvastatin depresses blood clotting by inhibiting activation of prothrombin, factor V, and factor XIII and by enhancing factor Va inactivation. Circulation 2001;103:2248-53.
16. Santos M T, Fuset M P, Ruano M, Moscardo A, Valles J. Effect of atorvastatin on platelet thromboxane A(2) synthesis in aspirin-treated patients with acute myocardial infarction. Am J Cardiol 2009;104:1618-23.
17. Habib A, Shamseddeen I, Nasrallah M S, Antoun T A, Nemer G, Bertoglio J et al. Modulation of COX-2 expression by statins in human monocytic cells. FASEB J 2007;21:1665-74.
18. FitzGerald G A. Cardiovascular pharmacology of nonselective nonsteroidal anti-inflammatory drugs and coxibs: clinical considerations. Am J Cardiol 2002;89:26D-32D.
19. Quyyumi A A. Effects of aspirin on endothelial dysfunction in atherosclerosis. Am J Cardiol 1998;82:31S-3S.
20. O'Driscoll G, Green D, Maiorana A, Stanton K, Colreavy F, Taylor R Improvement in endothelial function by angiotensin-converting enzyme inhibition in non-insulin-dependent diabetes. J Am Coll Cardiol 1999;33:1506-11.
21. Singh R B, Pella D, Neki N S, Chandel J P, Rastogi S, Mori H et al. Mechanisms of acute myocardial infarction study (MAMIS). Biomed Pharmacother 2004;58(Suppl 1):S111-S115.
22. Ghiadoni L, Donald A E, Cropley M, Mullen M J, Oakley G, Taylor M et al. Mental stress induces transient endothelial dysfunction in humans. Circulation 2000;102:2473-78.
23. Di Sciascio G, Patti G, Pasceri V, Gaspardone A, Colonna G, Montinaro A. Efficacy of atorvastatin reload in patients on chronic statin therapy undergoing percutaneous coronary intervention: results of the ARMYDA-RECAPTURE (Atorvastatin for Reduction of Myocardial Damage During Angioplasty). J Am Coll Cardiol 2009;54:558-65.
24. Briguori C, Visconti G, Focaccio A, Golia B, Chieffo A, Castelli A et al. Novel Approaches for Preventing or Limiting Events (Naples) II Trial: Impact of a single high loading dose of atorvastatin on periprocedural myocardial infarction. J Am Coll Cardiol 2009;54:2157-63.
25. Briguori C, Colombo A, Airoldi F, Violante A, Focaccio A, Balestrieri P et al. Statin administration before percutaneous coronary intervention: impact on periprocedural myocardial infarction. Eur Heart J, 2004;25:1822-28.
26. Zhang F, Dong L, Ge J. Effect of statins pretreatment on periprocedural myocardial infarction in patients undergoing percutaneous coronary intervention: a meta-analysis. Ann Med 2010;42:171-77.
27. Winchester D E, Wen X, Xie L, Bavry A A. Evidence of pre-procedural statin therapy a meta-analysis of randomized trials. J Am Coll Cardiol 2010;56:1099-109.
28. Fonarow G C, Wright R S, Spencer F A, Fredrick P D, Dong W, Every N et al. Effect of statin use within the first 24 hours of admission for acute myocardial infarction on early morbidity and mortality. Am J Cardiol 2005;96:611-16.
29. Patti G, Pasceri V, Colonna G, Miglionico M, Fischetti D, Sardella G et al. Atorvastatin pretreatment improves outcomes in patients with acute coronary syndromes undergoing early percutaneous coronary intervention: results of the ARMYDA-ACS randomized trial. J Am Coll Cardiol 2007;49:1272-78.
30. Lenderink T, Boersma E, Gitt A K, Zeymer U, Wallentin L, Van De W F et al. Patients using statin treatment within 24 h after admission for ST-elevation acute coronary syndromes had lower mortality than non-users: a report from the first Euro Heart Survey on acute coronary syndromes. Eur Heart J, 2006;27:1799-804.
31. Aronow H D, Topol E J, Roe M T, Houghtaling P L, Wolski K E, Lincoff A M et al. Effect of lipid-lowering therapy on early mortality after acute coronary syndromes: an observational study. Lancet 2001;357:1963-68.
32. Yun K H, Jeong M H, Oh S K, Rhee S J, Park E M, Lee E M et al. The beneficial effect of high loading dose of rosuvastatin before percutaneous coronary intervention in patients with acute coronary syndrome. Int J Cardiol 2009;137:246-51.
33. Kontopoulos A G, Athyros V G, Papageorgiou A A, Boudoulas H. Effect of quinapril or metoprolol on circadian sympathetic and parasympathetic modulation after acute myocardial infarction. Am J Cardiol 1999;84:1164-69.
34. Wilson P W, D'Agostino R B, Levy D, Belanger A M, Silbershatz H, Kannel W B. Prediction of coronary heart disease using risk factor categories. Circulation 1998;97:1837-47.
35. Hippisley-Cox J, Coupland C. Effect of combinations of drugs on all cause mortality in patients with ischaemic heart disease: nested case-control analysis. BMJ 2005;330:1059-63.
36. Thygesen K, Alpert J S, White H D, Jaffe A S, Apple F S, Galvani M et al. Universal Definition of Myocardial Infarction. Circulation 2007;116:2634-53.
37. Reichlin T, Irfan A, Twerenbold R, Reiter M, Hochholzer W, Burkhalter H et al. Utility of Absolute and Relative Changes in Cardiac Troponin Concentrations in the Early Diagnosis of Acute Myocardial Infarction. Circulation 2011;124:136-45.
38. Cannon C P, Shah S, Dansky H M, Davidson M, Brinton E A, Gotto A M J et al. Safety of anacetrapib in patients with or at high risk for coronary heart disease. N Engl J Med 2010;363:2406-15.
39. Nicholls S J, Tazcu E M, Brennan D M, Tardif J C, Nissen S E. Cholesteryl ester transfer protein inhibition, high-density lipoprotein raising, and progression of coronary atherosclerosis: insights from ILLUSTRATE (Investigation of Lipid Level Management Using Coronary Ultrasound to Assess Reduction of Athersclerosis by CETP Inhibition and HDL Elevation). Circulation 2008;118:2506-14.
40. Nicholls S J, Brewer H B, Kastelein J J, Kreuger K A, Wang M, Shao M et al. Effects of the CETP inhibitor evacetrapib administrated as monotherapy or in combination with statins on HDL and LDL cholesterol: A randomized controlled trial. JAMA 2011;306: 2099-109.
41. Raal, F, Scott, R, Somaratne R, Bridges I, Li G, Wasserman S M, Stein E A. Low-density lipoprotein cholesterol-lowering effects of AMG 145, a monoclonal antibody to proprotein convertase subtilisin/Kexin type 9 serine protease in patients with heterozygous familial hypercholesterolemia: The reduction of LDL-C with PCSK9 inhibition in heterozygous familial hypercholesterolemia disorder (RUTHERFORD) randomized trial.
42. Stein E A, Gipe D, Gergeron J, Gaudet D, Weiss R, Dufour R, Wu R, Pordy R. Effect of a monoclonal antibody to PCSK9, REGN727/SAR236553, to reduce low-density lipoprotein cholesterol in patients with heterozygous familial hypercholesterolaemia on stable statin dose with or without ezetimibe therapy: a phase 2 randomised controlled trial. Lancet 2012;380:29-36.

This invention may be embodied in other forms or carried out in other ways without departing from the spirit or essential characteristics thereof. The present disclosure is therefore to be considered as in all aspects illustrate and not restrictive, and all changes which come within the meaning and range of equivalency are intended to be embraced therein.

Various publications are cited herein, the contents of which are hereby incorporated by reference in their entireties.

1. A method for identifying an investigative drug which is capable of reducing the risk of a myocardial infarction in a subject or determining whether an investigative drug increases the risk of a myocardial infarction in a subject, comprising:
(a) administering the investigative drug and optionally a statin to a test group of subjects, wherein the test group comprises subjects who are undergoing elective PCI or have ACS/acute myocardial infarction;
(b) administering a statin to a control group of subjects, wherein the test group has been administered an investigative drug and statin or administering a placebo to a control group of subject wherein the test group has been administered an investigative drug alone, wherein the control group comprises subjects who are undergoing elective PCI or have ACS/acute infarction;
(c) comparing the subsequent incidences of myocardial infarctions in the subjects who had PCI in the test group with those had PCI in the control group, and (d) comparing the subsequent incidences of short-term mortality and/or status of myocardial infarctions in the subjects who had ACS/acute infarction in the test group with that of those who had ACS/acute infarction in the control group;
wherein if there are significantly less incidences of myocardial infarctions in the subjects who had PCI as compared to those in the control group and/or significantly less incidences of short-term mortality and/or improved status of myocardial infarction in the subjects who had ACS/acute myocardial infarction in the test group as compared to those in the control group, then the investigative drug is capable of preventing myocardial infarctions or
wherein if there are significantly more incidences of myocardial infarctions in the subjects who had PCI as compared to those in the control group and/or significantly more incidences of short-term mortality in the subjects who had ACS/acute myocardial infarction and/or if status of myocardial infarction has deteriorated in the test group as compared to those in the control group, then the investigative drug increases the risk of a myocardial infarction.

2. The method of claim 1, wherein the investigative drug is a CETP inhibitor or PCSK9 agent.

3. The method of claim 1, wherein comparing the subsequent incidences of myocardial infarctions in the subjects who are undergoing PCI in the test group with those who are undergoing PCI in the control group is performed at least about one day after the PCI.

4. The method of claim 3, wherein the comparing is performed two or more days after the PCI.

5. The method of claim 1, wherein comparing the subsequent incidences of short-term mortality in the subjects who suffered from ACS/acute infarction with that of those who suffered from ACS/acute myocardial infarction in the control group is performed at least about one week after the ACS/acute infarction.

6. The method of claim 5, wherein the comparing is performed at least about two weeks after the ACS/acute myocardial infarction.

7. The method of claim 5, wherein the comparing is performed at least about one month after the ACS/acute myocardial infarction.

8. The method of claim 1, wherein the test group and the control group are given about the same dose of the statin.

9. The method of claim 1, wherein the test group and control group undergoing elective PCI are administered a statin in advance of the PCI.

10. The method of claim 1, wherein the test group undergoing elective PCI are administered the investigative drug in advance of the PCI.

11. The method of claim 9, wherein the test group undergoing elective PCI are administered the investigative drug in advance of the PCI.

12. A method of reducing the risk of infarctions and ischemic heart disease in a subject comprising administering a CETP inhibitor or PCSK9 agent identified as an effective drug using the method of claim 2.

13. The method according to claim 12, wherein said subject has undergone elective PCI within six months of administration of CETP inhibitor or PCSK9 agent and/or has had ACS/acute infarction within six months of administration of said CETP inhibitor or PCSK9 agent.

14. A method for modulating and/or treating infarctions and ischemic heart disease in a subject in need thereof comprising:
(a) identifying a drug capable of modulating and/or treating infarctions and ischemic heart disease comprising:
(i) administering an investigative drug and optionally a statin to a test group of subjects, wherein the test group comprises subjects who are undergoing elective PCI or have ACS/acute myocardial infarction;
(ii) administering a statin to a control group of subjects, wherein the test group has been administered an investigative drug and statin or administering a placebo to a control group of subject wherein the test group has been administered an investigative drug alone, wherein the control group comprises subjects who are undergoing elective PCI or have ACS/acute infarction;
(iii) comparing the subsequent incidences of myocardial infarctions in the subjects who had PCI in the test group with those had PCI in the control group, and
(iv) comparing the subsequent indices of short-term mortality and/or status of myocardial infarction in the subjects who had ACS/acute infarction with that of those who had ACS/acute infarction in the control group;
wherein if there are significantly less incidences of myocardial infarctions in the subjects who had PCI in the test group as compared to those in the control group and/or significantly less incidences of short-term mortality and/or improved status of mycocardial infarctions in the subjects who had ACS/acute myocardial infarction in the test group as compared to those in the control group, then the investigative drug is capable of preventing myocardial infarctions.
(b) administering said identified drug in an amount effective to modulating and/or treating infarctions and ischemic heart disease.

15. The method according to claim 14, wherein said drug is administered in combination with another substance used to treat infarctions and ischemic heart disease.

16. The method according to claim 15, wherein said other substance is a statin.

17. The method according to claim 1, wherein status of myocardial infarction is evaluated by determining the level of cardiac enzymes in test and control subjects.

18. The method according to claim 14, wherein status of myocardial infarction is evaluated by determining the level of cardiac enzymes in test and control subjects.

19. The method according to claim 17, wherein the status of myocardial infarction is improved if there is a significant reduction in the level of cardiac enzymes in test subjects as compared to control subjects.

20. The method according to claim 18, wherein the status of myocardial infarction is improved if there is a significant reduction in the level of cardiac enzymes in test subjects as compared to control subjects.
